(19) United States
(12) Patent Application Publication
(10) Pub. No.: US 2016/0143862 A1
(43) Pub. Date: May 26, 2016

(54) METHOD OF USING BETA-IONONE TO TREAT AND PREVENT DISEASE INDUCED BY PROPIONIBACTERIUM ACNES

(71) Applicant: NATIONAL TAIWAN NORMAL UNIVERSTITY, TAIPEI CITY (TW)

(72) Inventors: PO-JUNG TSAI, NEW TAIPEI CITY (TW); WEN-CHENG HUANG, NEW TAIPEI CITY (TW); YA-HSIN SHIH, TAINAN CITY (TW)

(21) Appl. No.: 14/633,170

(22) Filed: Feb. 27, 2015

(30) Foreign Application Priority Data
Nov. 21, 2014 (TW) 103140354

(51) Int. Cl. A61K 31/12

(52) U.S. Cl. CPC A61K 31/12

(57) ABSTRACT

A method of using beta-ionone, E)-4-(2,6,6-trimethylcyclohex-1-en-1-yl)but-3-en-2-one, treats and prevents diseases otherwise induced by Propionibacterium acnes and thus effectively reduces inflammatory responses otherwise caused by Propionibacterium acnes.

### CROSS-REFERENCE TO RELATED APPLICATION

This non-provisional application claims priority under 35 U.S.C. §119(a) on Patent Application No(s).103140354 filed in Taiwan, R.O.C. on Nov. 21, 2014, the entire contents of which are hereby incorporated by reference.

### FIELD OF THE INVENTION

The present invention relates to methods of using beta-ionone, (3E)-4-(2,6,6-trimethylcyclohex-1-en-1-yl)but-3-en-2-one, to treat and prevent diseases induced by Propionibacterium acnes, and more particularly, to a method of using beta-ionone, (3E)-4-(2,6,6-trimethylcyclohex-1-en-1-yl)but-3-en-2-one, to reduce inflammatory responses otherwise caused by Propionibacterium acnes.

### BACKGROUND OF THE INVENTION

Propionibacterium acnes, a gram-positive bacterium, is a major pathogenic factor in acne vulgaris, and is possibly associated with allergic alveolitis, rheumatoid arthritis, infectious keratitis, corneal ulcer, and endophthalmitis, peritonitis, and prostate inflammation, and fulminant hepatic failure. Acne vulgaris, a chronic inflammatory disease of the pilosebaceous follicles, typically affects the face, neck, chest, upper limbs, and back. Acne vulgaris occurs for various reasons, including excessive sebaceous gland secretion, abnormal follicular keratinization, bacterial colonization, and host inflammatory responses. In the development of inflammatory lesions, growth factors, cytokines and hormones produced locally in the pilosebaceous unit play an important role. In acne lesions, these mediators are found to be up-regulated and activated immune cells are detected in inflammatory acne lesions. Propionibacterium acnes activates immune cells with the consecutive secretion of various pro-inflammatory cytokines, including IL-1, IL-6, IL-8 and TNFα. In particular, Interleukin-8 (IL-8) appears to play a key role in the pathogenesis of acne. Conventional treatment of acne vulgaris falls into two categories, that is, topical treatment and systemic treatment. Mild to moderate acne vulgaris is treated with related medications for external use, whereas severe acne vulgaris is treated systemically. At present, topical medications favored by dermatologists include benzoyl peroxide, salicylic acid, azelaic acid, retinoic acid, and antibiotics, whereas severe acne vulgaris is treated with systemic medications, including oral antibiotics and oral retinoic acid, as well as by hormone therapy. However, the aforesaid medications have side effects, such as dry skin, prickly skin, peeling skin, and drug resistance. Accordingly, it is imperative to develop a novel medication for treating acne vulgaris.

### SUMMARY OF THE INVENTION

In view of the aforesaid drawbacks of the prior art, it is an objective of the present invention to provide a method of using beta-ionone, (3E)-4-(2,6,6-trimethylcyclohex-1-en-1-yl)but-3-en-2-one, to treat and prevent diseases otherwise induced by Propionibacterium acnes with a view to solving the problems with the side effects of the conventional medications for treating acne vulgaris.

Beta-ionone exists widely in various vegetables, fruits, and plants. Beta-ionone is a class of cyclic terpenoids occurring in essential oils exhibiting a sweet floral scent reminiscent of violets.

Beta-ionone not only applies to the production of synthetic fragrances, but, as revealed by related literature, also plays an important role in inhibiting bacteria, cancer growth, and mouse microglia inflammatory responses induced by lipopolysaccharide (LPS) of gram-negative bacteria. In view of this, the present invention provides a method of using beta-ionone in treating and preventing related diseases and inflammatory responses induced by Propionibacterium acnes.

In order to achieve the above and other objectives, the present invention provides a method of using beta-ionone, (3E)-4-(2,6,6-trimethylcyclohex-1-en-1-yl)but-3-en-2-one, to treat and prevent diseases otherwise induced by Propionibacterium acnes. Regarding the aforesaid method, the beta-ionone reduces inflammatory responses caused by Propionibacterium acnes.

Regarding the aforesaid method, diseases induced by Propionibacterium acnes is treated and prevented by inhibiting the release of a proinflammatory cytokinefrom leukocytes.

Regarding the aforesaid method, the proinflammatory factor is interleukin-8 (IL-8).

Regarding the aforesaid method, an effective concentration of the beta-ionone is less than 50 μM.

Regarding the aforesaid method, the beta-ionone functions as an anti-inflammation agent against P. acnes.

Accordingly, the present invention provides a method of using beta-ionone to treat and prevent diseases otherwise induced by Propionibacterium acnes with a view to reducing inflammatory responses otherwise caused by Propionibacterium acnes and alleviating the symptoms of inflammatory acne.

### BRIEF DESCRIPTION OF THE DRAWINGS

Objectives, features, and advantages of the present invention are hereunder illustrated with specific embodiments in conjunction with the accompanying drawings, in which:

FIG. 1 is a bar chart of the test result of the effect of different concentrations of beta-ionone on the cytotoxicityof THP-1 cells according to embodiment 1 of present invention;

FIG. 2 is a bar chart of the test result of the effect of different concentrations of beta-ionone on P. acnes-induced IL-8 production by THP-1 cells according to embodiment 1 of present invention;

FIG. 3 is a graph of the thickness of mice's ears measured according to embodiment 2 of present invention;

FIG. 4 is a graph of the weight of biopsies from mice's ears measured according to embodiment 2 of present invention;

FIG. 5 is a histological picture taken, at 100-fold magnification, of a biopsy of mouse's ear in a PBS-control group according to embodiment 2 of present invention;

FIG. 6 is a histological picture taken, at 100-fold magnification, of a biopsy of mouse's ear in a P. acne group according to embodiment 2 of present invention;

FIG. 7 is a histological picture taken, at 100-fold magnification, of a biopsy of mouse's ear in a P. acnes+beta-ionone group according to embodiment 2 of present invention;

FIG. 8 is a histological picture taken of neutrophils, at 1000-fold magnification, of a biopsy of mouse's ear in a P. acnes group according to embodiment 2 of present invention; and

FIG. 9 is a picture taken of red, swollen ears of a mouse according to embodiment 2 of present invention.

### DETAILED DESCRIPTION OF THE PREFERRED EMBODIMENTS

Experiments are conducted in vitro and in vivo according to various embodiments of the present invention.

### Embodiment 1

### In Vitro Study

Preparation of Culture Fluid

In this embodiment, the in vitro study involves using RPMI 1640 culture medium which contains 2 μM of L-glutamine, 4.5 g/L of glucose, 10 μM of 4-(2-hydroxyethyl)-1-piperazineethanesulfonic acid (HEPES), and 1.0 mM of sodium pyruvate.

Human monocytic THP-1 cells (BCRC 60430, purchased from the Bioresource Collection and Research Center, Hsinchu, Taiwan) were cultured in RPMI 1640 medium and supplemented with 10% of fetal bovine serum, 0.05 mM of 2-mercaptoethanol, and 100 units/mL of penicillin G sodium, 100 μg/mL of streptomycin sulfate, and 250 ng/mL of amphotericin B at 37° C. in a 5% CO₂ humidified environment.

Propionibacterium acnes (BCRC 10723, purchased from the the Bioresource Collection and Research Center, Hsinchu, Taiwan) was cultured in a brain heart infusion (BHI) broth. The bacteria were cultured in an anaerobic atmosphere using BBL GasPak systems.

First, a dosage test is conducted to determine that beta-ionone at the concentrations of 10, 20, 50 μM does not have any cytotoxic effect on human monocytic THP-1 cells. Second, the inhibitory effect of beta-ionone on IL-8 production is carried out on human monocyticTHP-1cells. THP-1 cells are seeded at 2×10⁵ cells/well in a 96-well plate with FBS-free RMPI 1640 medium, and are stimulated with Propionibacterium acnes (multiplicity of infection=75) alone or in combination with various concentrations of 10, 20, 50 μM of beta-ionone for a 24-h incubation. Cell-free supernatants are then collected, and IL-8 concentration is analyzed with the enzyme immunoassay kit (Invitrogen)

Statistically Analysis

In an embodiment of the present invention, each experiment is independently carried out thrice, and the results are presented in terms of mean±standard deviation and analyzed with SPSS17.0 software, including Student's t-test, One-Way ANOVA, and Least Significant Difference (LSD), wherein p<0.05 (*), p<0.01 (**), and p<0.001 (***) indicate a significant statistical difference.

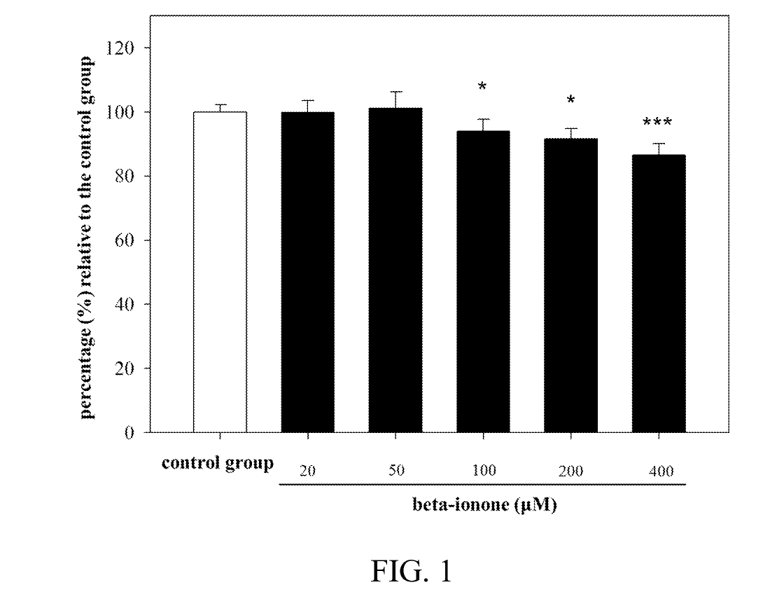

FIG. 1 shows the cytotoxicity effect of variousconcentrations of beta-ionone on THP-1 cells, wherein the THP-1 cells not treated with beta-ionone functions as a control group, such that the cellular viability of THP-1 cells treated with beta-ionone at different concentrations are compared with the cellular viabiltyof the control group. The test result shows that beta-ionone at a concentration of 100 μM or higher has a cytotoxic effect, with p<0.05 (*), p<0.001 (***).

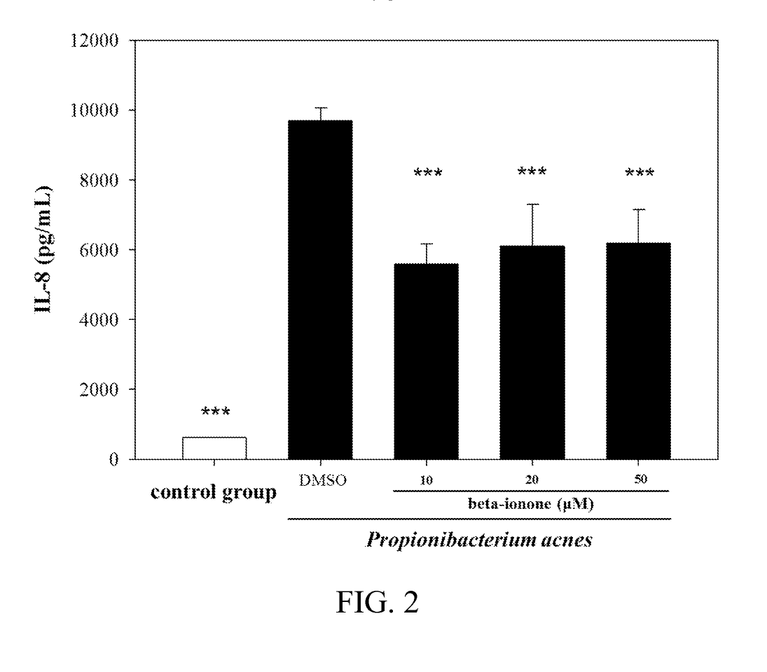

FIG. 2 shows the inhibitory effect of beta-ionone on P. acnes-induced IL-8 production by THP-1 cells, wherein the THP-1 without treatment with P. acnes and beta-ionone functions as the control group, and the experimental groups are the THP-1 cells treated with DMSO and beta-ionone at different concentrations. The result shows that beta-ionone at different concentrations of 10, 20, 50 μM demonstrates a marked inhibitive effect, with p<0.001 (***).

In this embodiment, IL-8 level is indicative of P. acnes-induced inflammatory responses. IL-8 is a chemokine produced by THP-1. IL-8 is also a chemoattractant involved in the recruitment of neutrophils, the predominant cell type in acne-related lesions.

### Embodiment 2

### Animal Experiment (In Vivo Study)

In the animal experiment of this embodiment, four-week-old male ICRmice were purchased from the Experimental Animal Center of the National Taiwan University. After 1 week of adaptation, the mice were randomly divided into three groups of six animals each.

The three groups of mice are, namely a blank control group of mice injected with a phosphate buffer solution (hereinafter referred to as “PBS-control”), an experimental group of mice injected with Propionibacterium acnes (hereinafter referred to as “P. acnes group”), and an experimental group of mice injected with Propionibacterium acnes and beta-ionone (hereinafter referred to as “P. acnes+beta-ionone group”). Mice were housed in plastic cages, under standard temperature-controlled conditions with a 12 h/12 h light-dark cycle and free access to food and water throughout the experiments.

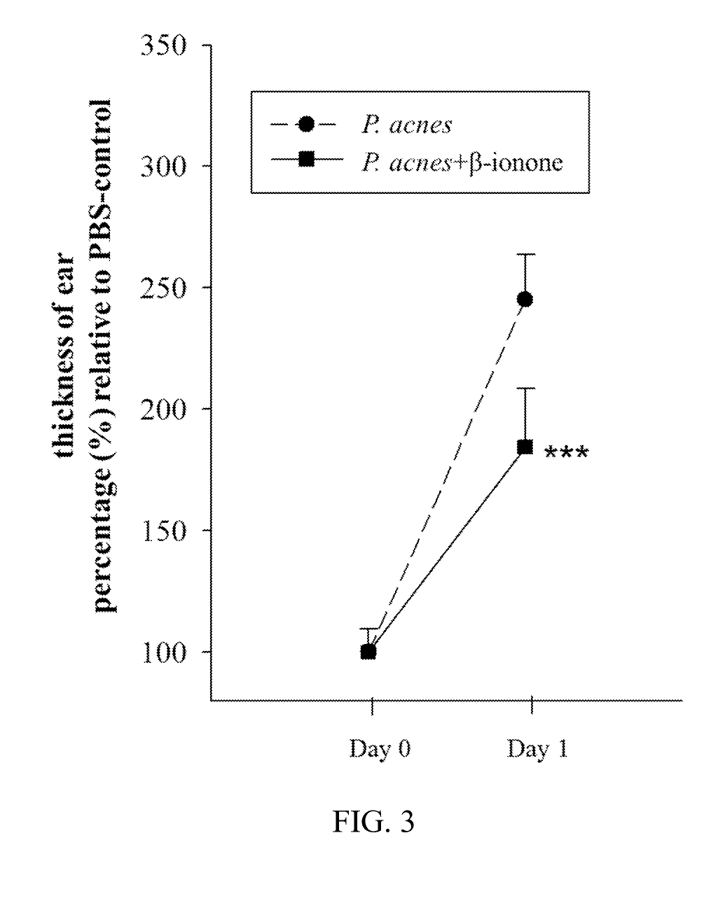

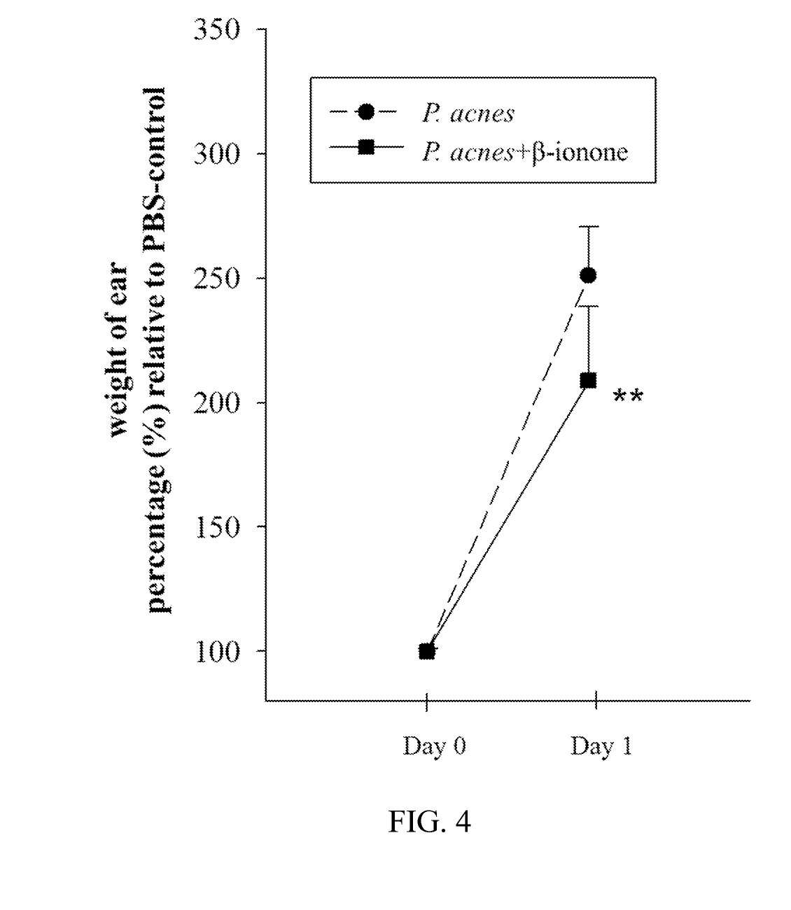

The experiments are each carried out by following the steps described below, so as to inject 10 L of liquid (phosphate buffer solution (PBS), P. acnes, or P. acnes+beta-ionone) into the mice' ears, respectively. (1) PBS-control: inject 10 μL of PBS (0.16% aqueous solution of phosphate, a saline solution, 0.14% Na₂HPO₄, and 0.02% KH₂PO₄) into the mice's left ears, but inject no solution or reagent to the mice's right ears. (2) P. acnes group: dissolve P. acnes in PBS to the extent that the bacterial content of the PBS is 6×10⁷ CFU/100 μL and then inject the 100 μL of bacteria-containing PBS into the mice's left ears and 100 μL of PBS into the mice's right ears. (3) P. acnes+beta-ionone group: inject 10 μL of P. acnes-containing solution (6×10⁷ CFU/10 μL) into the mice's left ears, and dissolve P. acnes and beta-ionone in PBS to thereby inject the PBS which contain 6×10⁷ CFU of P. acnes and 50 μg/10 μL of beta-ionone into the mice's right ears. Mercy killing is carried out to all the mice 24 hours after the aforesaid injections, and then the ear's swelling induced by P. acnes was quantified as the percentage increases in the thickness and weight of the left ear biopsy. The thickness of the mice's ears is measured with a micrometer caliper. Furthermore, a biopsy is performed by removing a piece of tissue 4 mm in diameter from the dead mice's ears with a biopsy puncher, and then the tissue specimens are weighed with a precise electronic scale. The measurements of the thickness and weight of the mice's ears are expressed in percentage when compared with the PBS control group, and the results are shown in FIG. 3 and FIG. 4, respectively. Referring to FIG. 3 and FIG. 4, the measurements of the thickness and weight of the mice's ears are indicative effect of beta-ionone on the P. acnes-induced neutrophils infiltration in the mice's ear and the swelling of the mice's ear, and the measurements show that beta-ionone is effective in reducing the redness, neutrophils infiltration, and swelling otherwise caused by P. acnes, where p<0.01 (**), and p<0.001 (***).

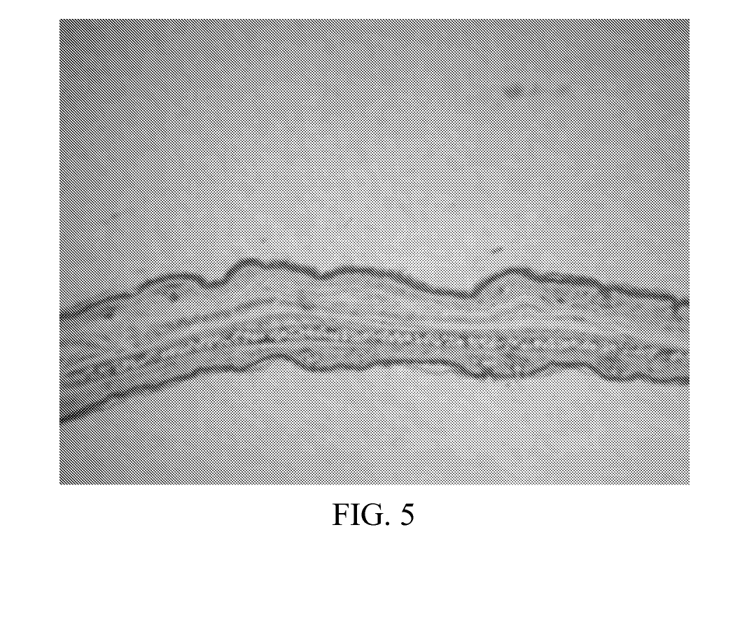

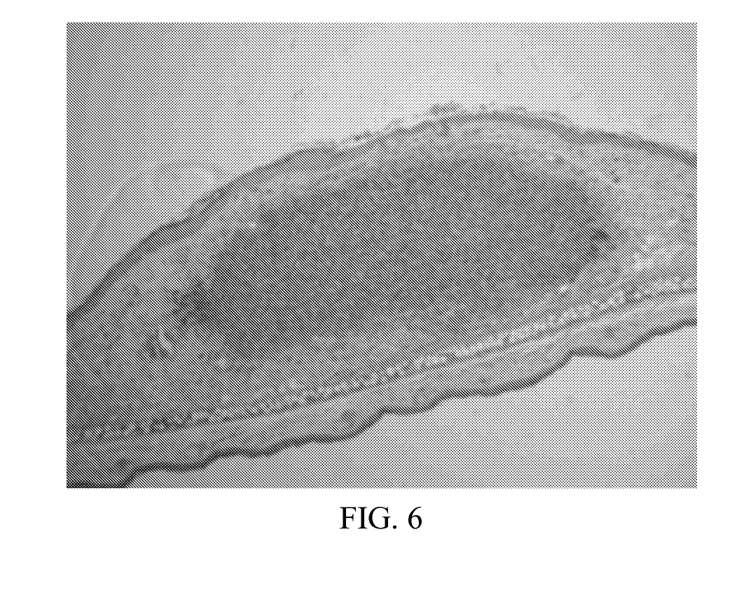

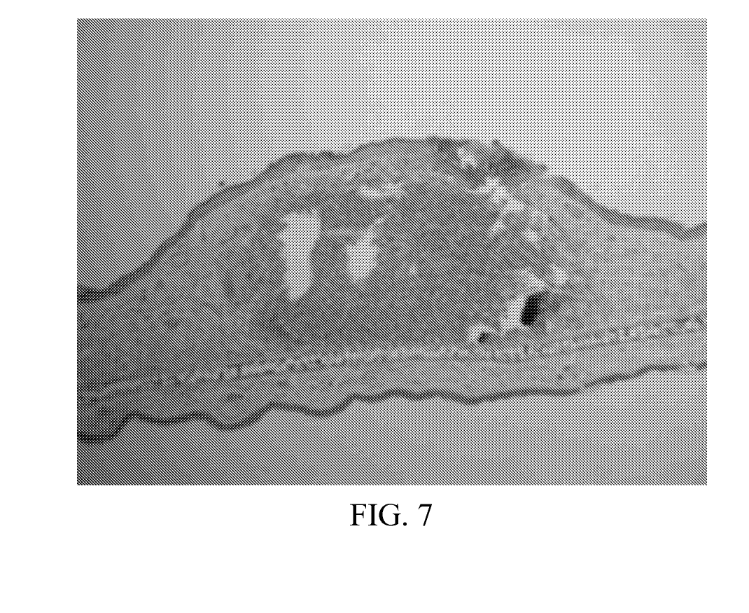

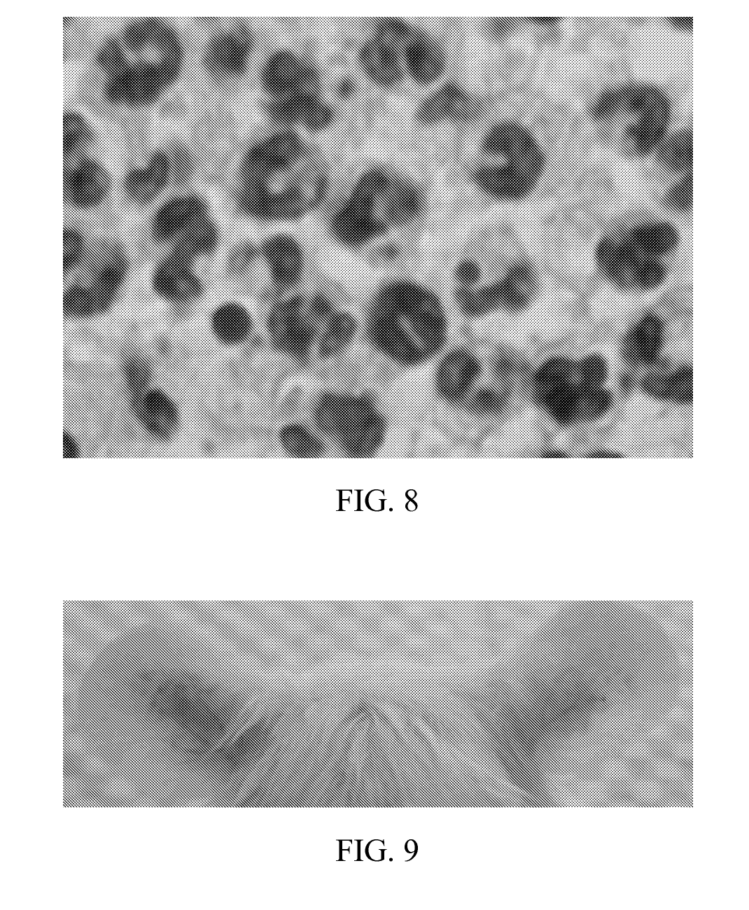

Histological pictures taken of the ears of the mice in the aforesaid three groups according to embodiment 2 of present invention are shown in FIG. 5 through FIG. 8. Referring to FIG. 5, there is shown a histological picture taken, at 100-fold magnification, of a mouse's ear in a PBS-control group according to embodiment 2 of present invention. Referring to FIG. 6, there is shown a histological picture taken, at 100-fold magnification, of a mouse's left ear in a P. acnes control group according to embodiment 2 of present invention. Referring to FIG. 7, there is shown a histological picture taken, at 100-fold magnification, of a mouse's right ear in a P. acnes+beta-ionone group according to embodiment 2 of present invention. Referring to FIG. 8, there is shown a histological picture taken, at 1000-fold magnification, of a mouse's ear in a P. acnes group according to embodiment 2 of present invention, confirming that neutrophils gather at the inflammatory lesion of the ear.

P. acnes+beta-ionone group: inject 10 μL of P. acnes-containing solution (6×10⁷ CFU/10 μL) into the mice's left ear, and dissolve P. acnes and beta-ionone in PBS to thereby inject the PBS which contain 6×10⁷ CFU of P. acnes and 50 μg/10 μL of beta-ionone into the mice's right ears. Then, compare the mice's left ears with the mice's right ears in terms of redness and swelling, 24 hours after the injections; and the results are shown in FIG. 9. Referring to FIG. 9, unlike the left ears which P. acnes is injected into, the right ears which both P. acnes and beta-ionone are injected into demonstrate significantly abated redness and swelling.

In conclusion, the results of experiments conducted in embodiment 1 and embodiment 2 prove that beta-ionone is effective in treating and preventing diseases otherwise induced by Propionibacterium acnes. Specifically speaking, beta-ionone reduces the inflammatory responses otherwise caused by Propionibacterium acnes and thus is effective in alleviating inflammatory acne vulgaris, treating and preventing the other inflammatory diseases otherwise induced by Propionibacterium acnes, such as allergic alveolitis, rheumatoid arthritis, eye infections (including infectious keratitis, corneal ulcer, and endophthalmitis), peritonitis, prostate inflammation, prostate cancer caused by inflammation, and acute or fulminant hepatic failure.

The present invention is disclosed above by preferred embodiments. However, persons skilled in the art should understand that the preferred embodiments are illustrative of the present invention only, but should not be interpreted as restrictive of the scope of the present invention. Hence, all equivalent modifications and replacements made to the aforesaid embodiments should fall within the scope of the present invention. Accordingly, the legal protection for the present invention should be defined by the appended claims.

What is claimed is:

1. A method of using beta-ionone,
(3E)-4-(2,6,6-trimethylcyclohex-1-en-1-yl)but-3-en-2-one, to treat and prevent diseases otherwise induced by Propionibacterium acnes.

2. The method of claim 1, wherein the beta-ionone reduces inflammatory responses caused by Propionibacterium acnes.

3. The method of claim 2, wherein diseases induced by Propionibacterium acnes is treated and prevented by inhibiting release of a proinflammatory chemokine from leukocytes.

4. The method of claim 3, wherein the proinflammatory factor is a cellularchemokine.

5. The method of claim 4, wherein the cellular chemokine is interleukin-8 (IL-8).

6. The method of claim 4, wherein an effective concentration of the beta-ionone is less than 50 μM.
